(19) United States
(12) Patent Application Publication
(10) Pub. No.: US 2023/0207140 A1
(43) Pub. Date: Jun. 29, 2023

(54) METHOD AND APPARATUS FOR PREVENTING SPREAD OF INFECTION

(71) Applicant: LG ELECTRONICS INC., Seoul (KR)

(72) Inventors: Jeongki Kim, Seoul (KR); Sanggook Kim, Seoul (KR); Jinsoo Choi, Seoul (KR)

(21) Appl. No.: 17/919,709

(22) PCT Filed: Apr. 12, 2021
(86) PCT No.: PCT/KR2021/004558

§ 371 Date: Oct. 18, 2022

Related U.S. Application Data
(60) Provisional application No. 63/014,134, filed on Apr. 23, 2020.

(51) Int. Cl. G16H 50/80; H04W 64/00

(52) U.S. Cl. CPC G16H 50/80; H04W 64/003

(57) ABSTRACT

A receiving smart device can receive, from an adjacent transmitting smart device, a signal including information about the transmitting smart device, and can transmit a report signal including position information about the transmitting smart device if the information about the transmission smart device is consistent with confirmed case information. The information about confirmed cases may include at least one of a medium access control (MAC) address of a smart device used by the confirmed cases, phone number information of the confirmed cases, and information related to criteria for judging a person in contact with the confirmed cases.

### BACKGROUND OF THE DISCLOSURE

### Field of the Disclosure

The present specification relates to a method of providing location information between smart devices for preventing the spread of infectious diseases.

### Related Art

Global environmental changes, caused by reckless development, global warming, and the like, are causing new crises that humans have never experienced before. In addition, due to globalization, diseases that previously occurred locally are now spreading all over the world in a short period of time. It is thought that human beings will have a huge impact on social and economic life due to more frequent disease outbreaks in the future.

For example, the world is currently experiencing an unprecedented catastrophe due to the COVID-19 virus that originated in one region. This has resulted in mass unemployment and the exhaustion of vast communal wealth.

In order to prevent the spread of the epidemic, a lot of manpower is required to identify the movement of the confirmed cases and to find out who has come into contact with them. In addition, in reality, it is difficult to completely understand the movement of the confirmed case. Therefore, there is a need for a method to solve these problems.

### SUMMARY

### Technical Solutions

The receiving smart device may acquire information about confirmed cases. The receiving smart device may receive, from a transmitting smart device, a first signal including first information of the transmitting smart device. The receiving smart device may transmit a report signal including the first information and location information at which the first signal was received, based on the first information being included in the information about confirmed cases.

Technical Effects

According to an example of the present specification, the smart device may acquire information about the confirmed case and determine whether or not a person has been exposed to a close distance from the confirmed case. In the case of exposure to a confirmed case at a close distance, since a report signal including location information of contact with the confirmed person can be transmitted, the movement of the confirmed case can be easily identified, and people who have come into contact with the confirmed case can be easily tracked.

### BRIEF DESCRIPTION OF THE DRAWINGS

FIG. 1 shows an example of a transmitting apparatus and/or receiving apparatus of the present specification.

FIG. 2 is a conceptual view illustrating the structure of a wireless local area network (WLAN).

FIG. 3 shows a modified example of a transmitting apparatus and/or a receiving apparatus of the present specification.

FIG. 4 is a diagram illustrating a method for preventing the spread of infectious disease according to an embodiment of the present specification.

FIG. 5 is a diagram illustrating an embodiment of a method for operating a receiving smart device.

FIG. 6 is a diagram illustrating an embodiment of a method of operating a transmitting smart device.

### DETAILED DESCRIPTION

In the present specification, “A or B” may mean “only A”, “only B” or “both A and B”. In other words, in the present specification, “A or B” may be interpreted as “A and/or B”. For example, in the present specification, “A, B, or C” may mean “only A”, “only B”, “only C”, or “any combination of A, B, C”.

A slash (/) or comma used in the present specification may mean “and/or”. For example, “A/B” may mean “A and/or B”. Accordingly, “A/B” may mean “only A”, “only B”, or “both A and B”. For example, “A, B, C” may mean “A, B, or C”.

In the present specification, “at least one of A and B” may mean “only A”, “only B”, or “both A and B”. In addition, in the present specification, the expression “at least one of A or B” or “at least one of A and/or B” may be interpreted as “at least one of A and B”.

In addition, in the present specification, “at least one of A, B, and C” may mean “only A”, “only B”, “only C”, or “any combination of A, B, and C”. In addition, “at least one of A, B, or C” or “at least one of A, B, and/or C” may mean “at least one of A, B, and C”.

In addition, a parenthesis used in the present specification may mean “for example”. Specifically, when indicated as “control information (EHT-signal)”, it may denote that “EHT-signal” is proposed as an example of the “control information”. In other words, the “control information” of the present specification is not limited to “EHT-signal”, and “EHT-signal” may be proposed as an example of the “control information”. In addition, when indicated as “control information (i.e., EHT-signal)”, it may also mean that “EHT-signal” is proposed as an example of the “control information”.

Technical features described individually in one figure in the present specification may be individually implemented, or may be simultaneously implemented.

The following example of the present specification may be applied to various wireless communication systems. For example, the following example of the present specification may be applied to a wireless local area network (WLAN) system. For example, the present specification may be applied to the IEEE 802.11a/g/n/ac standard or the IEEE 802.11ax standard. In addition, the present specification may also be applied to the newly proposed EHT standard or IEEE 802.11be standard. In addition, the example of the present specification may also be applied to a new WLAN standard enhanced from the EHT standard or the IEEE 802.11be standard. In addition, the example of the present specification may be applied to a mobile communication system. For example, it may be applied to a mobile communication system based on long term evolution (LTE) depending on a 3^{rd} generation partnership project (3GPP) standard and based on evolution of the LTE. In addition, the example of the present specification may be applied to a communication system of a 5G NR standard based on the 3GPP standard.

Hereinafter, in order to describe a technical feature of the present specification, a technical feature applicable to the present specification will be described.

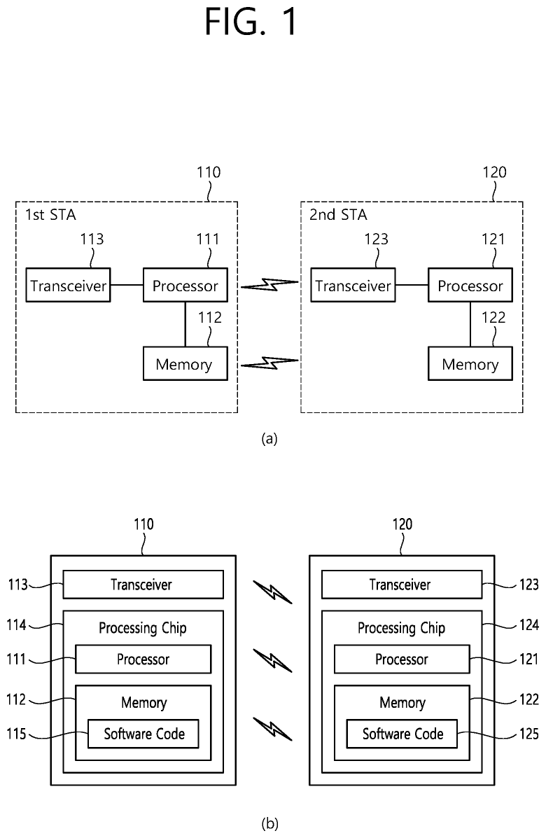

FIG. 1 shows an example of a transmitting apparatus and/or receiving apparatus of the present specification.

In the example of FIG. 1, various technical features described below may be performed. FIG. 1 relates to at least one station (STA). For example, STAs 110 and 120 of the present specification may also be called in various terms such as a mobile terminal, a wireless device, a wireless transmit/receive unit (WTRU), a user equipment (UE), a mobile station (MS), a mobile subscriber unit, or simply a user. The STAs 110 and 120 of the present specification may also be called in various terms such as a network, a base station, a node-B, an access point (AP), a repeater, a router, a relay, or the like. The STAs 110 and 120 of the present specification may also be referred to as various names such as a receiving apparatus, a transmitting apparatus, a receiving STA, a transmitting STA, a receiving device, a transmitting device, or the like.

For example, the STAs 110 and 120 may serve as an AP or a non-AP. That is, the STAs 110 and 120 of the present specification may serve as the AP and/or the non-AP.

The STAs 110 and 120 of the present specification may support various communication standards together in addition to the IEEE 802.11 standard. For example, a communication standard (e.g., LTE, LTE-A, 5G NR standard) or the like based on the 3GPP standard may be supported. In addition, the STA of the present specification may be implemented as various devices such as a mobile phone, a vehicle, a personal computer, or the like. In addition, the STA of the present specification may support communication for various communication services such as voice calls, video calls, data communication, and self-driving (autonomous-driving), or the like.

The STAs 110 and 120 of the present specification may include a medium access control (MAC) conforming to the IEEE 802.11 standard and a physical layer interface for a radio medium.

The STAs 110 and 120 will be described below with reference to a sub-figure (a) of FIG. 1.

The first STA 110 may include a processor 111, a memory 112, and a transceiver 113. The illustrated process, memory, and transceiver may be implemented individually as separate chips, or at least two blocks/functions may be implemented through a single chip.

The transceiver 113 of the first STA performs a signal transmission/reception operation. Specifically, an IEEE 802.11 packet (e.g., IEEE 802.11a/b/g/n/ac/ax/be, etc.) may be transmitted/received.

For example, the first STA 110 may perform an operation intended by an AP. For example, the processor 111 of the AP may receive a signal through the transceiver 113, process a reception (RX) signal, generate a transmission (TX) signal, and provide control for signal transmission. The memory 112 of the AP may store a signal (e.g., RX signal) received through the transceiver 113, and may store a signal (e.g., TX signal) to be transmitted through the transceiver.

For example, the second STA 120 may perform an operation intended by a non-AP STA. For example, a transceiver 123 of a non-AP performs a signal transmission/reception operation. Specifically, an IEEE 802.11 packet (e.g., IEEE 802.11a/b/g/n/ac/ax/be packet, etc.) may be transmitted/received.

For example, a processor 121 of the non-AP STA may receive a signal through the transceiver 123, process an RX signal, generate a TX signal, and provide control for signal transmission. A memory 122 of the non-AP STA may store a signal (e.g., RX signal) received through the transceiver 123, and may store a signal (e.g., TX signal) to be transmitted through the transceiver.

For example, an operation of a device indicated as an AP in the specification described below may be performed in the first STA 110 or the second STA 120. For example, if the first STA 110 is the AP, the operation of the device indicated as the AP may be controlled by the processor 111 of the first STA 110, and a related signal may be transmitted or received through the transceiver 113 controlled by the processor 111 of the first STA 110. In addition, control information related to the operation of the AP or a TX/RX signal of the AP may be stored in the memory 112 of the first STA 110. In addition, if the second STA 120 is the AP, the operation of the device indicated as the AP may be controlled by the processor 121 of the second STA 120, and a related signal may be transmitted or received through the transceiver 123 controlled by the processor 121 of the second STA 120. In addition, control information related to the operation of the AP or a TX/RX signal of the AP may be stored in the memory 122 of the second STA 120.

For example, in the specification described below, an operation of a device indicated as a non-AP (or user-STA) may be performed in the first STA 110 or the second STA 120. For example, if the second STA 120 is the non-AP, the operation of the device indicated as the non-AP may be controlled by the processor 121 of the second STA 120, and a related signal may be transmitted or received through the transceiver 123 controlled by the processor 121 of the second STA 120. In addition, control information related to the operation of the non-AP or a TX/RX signal of the non-AP may be stored in the memory 122 of the second STA 120. For example, if the first STA 110 is the non-AP, the operation of the device indicated as the non-AP may be controlled by the processor 111 of the first STA 110, and a related signal may be transmitted or received through the transceiver 113 controlled by the processor 111 of the first STA 110. In addition, control information related to the operation of the non-AP or a TX/RX signal of the non-AP may be stored in the memory 112 of the first STA 110.

In the specification described below, a device called a (transmitting/receiving) STA, a first STA, a second STA, a STA1, a STA2, an AP, a first AP, a second AP, an AP1, an AP2, a (transmitting/receiving) terminal, a (transmitting/receiving) device, a (transmitting/receiving) apparatus, a network, or the like may imply the STAs 110 and 120 of FIG. 1. For example, a device indicated as, without a specific reference numeral, the (transmitting/receiving) STA, the first STA, the second STA, the STA1, the STA2, the AP, the first AP, the second AP, the AP1, the AP2, the (transmitting/receiving) terminal, the (transmitting/receiving) device, the (transmitting/receiving) apparatus, the network, or the like may imply the STAs 110 and 120 of FIG. 1. For example, in the following example, an operation in which various STAs transmit/receive a signal (e.g., a PPDU) may be performed in the transceivers 113 and 123 of FIG. 1. In addition, in the following example, an operation in which various STAs generate a TX/RX signal or perform data processing and computation in advance for the TX/RX signal may be performed in the processors 111 and 121 of FIG. 1. For example, an example of an operation for generating the TX/RX signal or performing the data processing and computation in advance may include: 1) an operation of determining/obtaining/configuring/computing/decoding/encoding bit information of a sub-field (SIG, STF, LTF, Data) included in a PPDU; 2) an operation of determining/configuring/obtaining a time resource or frequency resource (e.g., a subcarrier resource) or the like used for the sub-field (SIG, STF, LTF, Data) included the PPDU; 3) an operation of determining/configuring/obtaining a specific sequence (e.g., a pilot sequence, an STF/LTF sequence, an extra sequence applied to SIG) or the like used for the sub-field (SIG, STF, LTF, Data) field included in the PPDU; 4) a power control operation and/or power saving operation applied for the STA; and 5) an operation related to determining/obtaining/configuring/decoding/encoding or the like of an ACK signal. In addition, in the following example, a variety of information used by various STAs for determining/obtaining/configuring/computing/decoding/decoding a TX/RX signal (e.g., information related to a field/subfield/control field/parameter/power or the like) may be stored in the memories 112 and 122 of FIG. 1.

The aforementioned device/STA of the sub-figure (a) of FIG. 1 may be modified as shown in the sub-figure (b) of FIG. 1. Hereinafter, the STAs 110 and 120 of the present specification will be described based on the sub-figure (b) of FIG. 1.

For example, the transceivers 113 and 123 illustrated in the sub-figure (b) of FIG. 1 may perform the same function as the aforementioned transceiver illustrated in the sub-figure (a) of FIG. 1. For example, processing chips 114 and 124 illustrated in the sub-figure (b) of FIG. 1 may include the processors 111 and 121 and the memories 112 and 122. The processors 111 and 121 and memories 112 and 122 illustrated in the sub-figure (b) of FIG. 1 may perform the same function as the aforementioned processors 111 and 121 and memories 112 and 122 illustrated in the sub-figure (a) of FIG. 1.

A mobile terminal, a wireless device, a wireless transmit/receive unit (WTRU), a user equipment (UE), a mobile station (MS), a mobile subscriber unit, a user, a user STA, a network, a base station, a Node-B, an access point (AP), a repeater, a router, a relay, a receiving unit, a transmitting unit, a receiving STA, a transmitting STA, a receiving device, a transmitting device, a receiving apparatus, and/or a transmitting apparatus, which are described below, may imply the STAs 110 and 120 illustrated in the sub-figure (a)/(b) of FIG. 1, or may imply the processing chips 114 and 124 illustrated in the sub-figure (b) of FIG. 1. That is, a technical feature of the present specification may be performed in the STAs 110 and 120 illustrated in the sub-figure (a)/(b) of FIG. 1, or may be performed only in the processing chips 114 and 124 illustrated in the sub-figure (b) of FIG. 1. For example, a technical feature in which the transmitting STA transmits a control signal may be understood as a technical feature in which a control signal generated in the processors 111 and 121 illustrated in the sub-figure (a)/(b) of FIG. 1 is transmitted through the transceivers 113 and 123 illustrated in the sub-figure (a)/(b) of FIG. 1. Alternatively, the technical feature in which the transmitting STA transmits the control signal may be understood as a technical feature in which the control signal to be transferred to the transceivers 113 and 123 is generated in the processing chips 114 and 124 illustrated in the sub-figure (b) of FIG. 1.

For example, a technical feature in which the receiving STA receives the control signal may be understood as a technical feature in which the control signal is received by means of the transceivers 113 and 123 illustrated in the sub-figure (a) of FIG. 1. Alternatively, the technical feature in which the receiving STA receives the control signal may be understood as the technical feature in which the control signal received in the transceivers 113 and 123 illustrated in the sub-figure (a) of FIG. 1 is obtained by the processors 111 and 121 illustrated in the sub-figure (a) of FIG. 1. Alternatively, the technical feature in which the receiving STA receives the control signal may be understood as the technical feature in which the control signal received in the transceivers 113 and 123 illustrated in the sub-figure (b) of FIG. 1 is obtained by the processing chips 114 and 124 illustrated in the sub-figure (b) of FIG. 1.

Referring to the sub-figure (b) of FIG. 1, software codes 115 and 125 may be included in the memories 112 and 122. The software codes 115 and 126 may include instructions for controlling an operation of the processors 111 and 121. The software codes 115 and 125 may be included as various programming languages.

The processors 111 and 121 or processing chips 114 and 124 of FIG. 1 may include an application-specific integrated circuit (ASIC), other chipsets, a logic circuit and/or a data processing device. The processor may be an application processor (AP). For example, the processors 111 and 121 or processing chips 114 and 124 of FIG. 1 may include at least one of a digital signal processor (DSP), a central processing unit (CPU), a graphics processing unit (GPU), and a modulator and demodulator (modem). For example, the processors 111 and 121 or processing chips 114 and 124 of FIG. 1 may be SNAPDRAGON™ series of processors made by Qualcomm®, EXYNOS™ series of processors made by Samsung®, A series of processors made by Apple®, HELIO™ series of processors made by MediaTek®, ATOM™ series of processors made by Intel® or processors enhanced from these processors.

In the present specification, an uplink may imply a link for communication from a non-AP STA to an SP STA, and an uplink PPDU/packet/signal or the like may be transmitted through the uplink. In addition, in the present specification, a downlink may imply a link for communication from the AP STA to the non-AP STA, and a downlink PPDU/packet/signal or the like may be transmitted through the downlink.

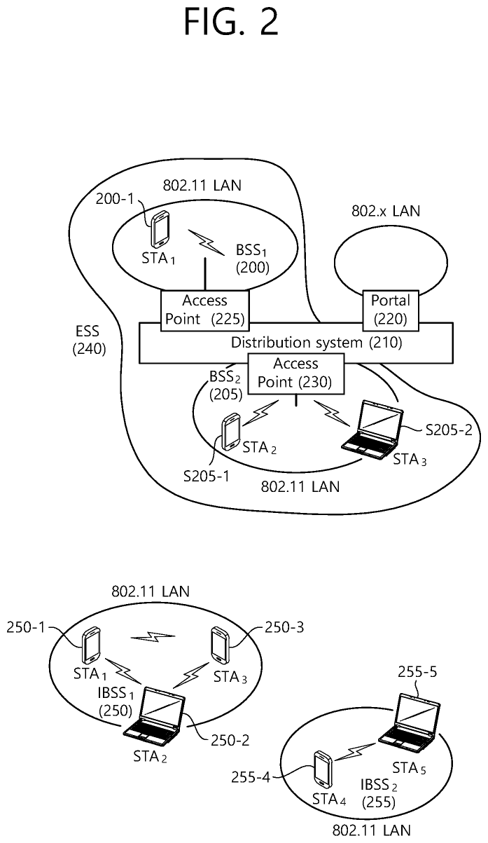

FIG. 2 is a conceptual view illustrating the structure of a wireless local area network (WLAN).

An upper part of FIG. 2 illustrates the structure of an infrastructure basic service set (BSS) of institute of electrical and electronic engineers (IEEE) 802.11.

Referring the upper part of FIG. 2, the wireless LAN system may include one or more infrastructure BSSs 200 and 205 (hereinafter, referred to as BSS). The BSSs 200 and 205 as a set of an AP and a STA such as an access point (AP) 225 and a station (STA1) 200-1 which are successfully synchronized to communicate with each other are not concepts indicating a specific region. The BSS 205 may include one or more STAs 205-1 and 205-2 which may be joined to one AP 230.

The BSS may include at least one STA, APs providing a distribution service, and a distribution system (DS) 210 connecting multiple APs.

The distribution system 210 may implement an extended service set (ESS) 240 extended by connecting the multiple BSSs 200 and 205. The ESS 240 may be used as a term indicating one network configured by connecting one or more APs 225 or 230 through the distribution system 210. The AP included in one ESS 240 may have the same service set identification (SSID).

A portal 220 may serve as a bridge which connects the wireless LAN network (IEEE 802.11) and another network (e.g., 802.X).

In the BSS illustrated in the upper part of FIG. 2, a network between the APs 225 and 230 and a network between the APs 225 and 230 and the STAs 200-1, 205-1, and 205-2 may be implemented. However, the network is configured even between the STAs without the APs 225 and 230 to perform communication. A network in which the communication is performed by configuring the network even between the STAs without the APs 225 and 230 is defined as an Ad-Hoc network or an independent basic service set (IBSS).

A lower part of FIG. 2 illustrates a conceptual view illustrating the IBSS.

Referring to the lower part of FIG. 2, the IBSS is a BSS that operates in an Ad-Hoc mode. Since the IBSS does not include the access point (AP), a centralized management entity that performs a management function at the center does not exist. That is, in the IBSS, STAs 250-1, 250-2, 250-3, 255-4, and 255-5 are managed by a distributed manner. In the IBSS, all STAs 250-1, 250-2, 250-3, 255-4, and 255-5 may be constituted by movable STAs and are not permitted to access the DS to constitute a self-contained network.

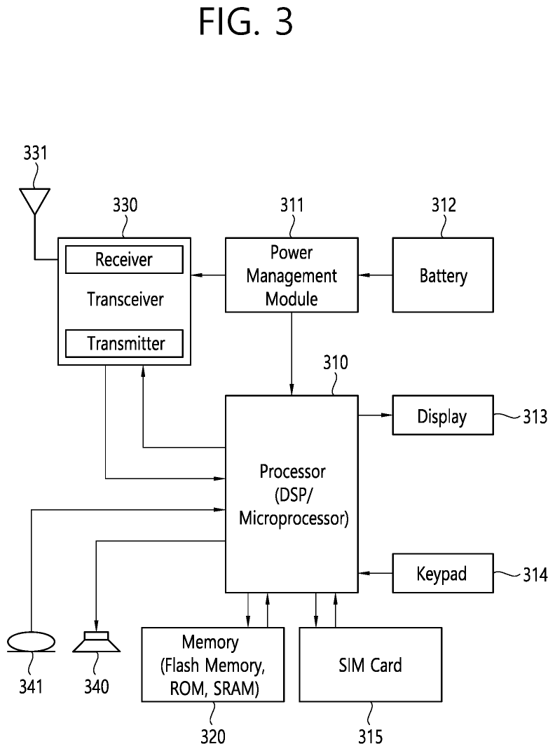

FIG. 3 shows a modified example of a transmitting apparatus and/or a receiving apparatus of the present specification.

Each device/STA of the sub-drawings (a)/(b) of FIG. 1 may be modified as shown in FIG. 3. The transceiver 330 of FIG. 3 may be the same as the transceivers 113 and 123 of FIG. 1. The transceiver 330 of FIG. 3 may include a receiver and a transmitter.

The processor 310 of FIG. 3 may be the same as the processors 111 and 121 of FIG. 1. Alternatively, the processor 310 of FIG. 3 may be the same as the processing chips 114 and

The memory 150 of FIG. 3 may be the same as the memories 112 and 122 of FIG. 1. Alternatively, the memory 150 of FIG. 3 may be a separate external memory different from the memories 112 and 122 of FIG. 1.

Referring to FIG. 3, the power management module 311 manages power for the processor 310 and/or the transceiver 330. The battery 312 supplies power to the power management module 311. The display 313 outputs the result processed by the processor 310. Keypad 314 receives input to be used by processor 310. The keypad 314 may be displayed on the display 313. SIM card 315 may be an integrated circuit used to securely store an international mobile subscriber identity (IMSI) and its associated keys used to identify and authenticate subscribers in mobile phone devices, such as mobile phones and computers.

Referring to FIG. 3, the speaker 340 may output a sound related result processed by the processor 310. Microphone 341 may receive sound related input to be used by processor 310.

This specification describes a method of implementing prevention of the spread of a catastrophic event using smart devices (for example, smart phones, smart watches, etc.) that have become close possessions in an individual's life. In particular, a method of implementing an operation suitable for the characteristics of a disaster (for example, an epidemic, etc.) using wireless access technologies provided by smart devices is considered as the main focus.

For wireless access used in this specification, representative access methods such as Wi-Fi (that is, wireless local area network, WLAN), Bluetooth Low Energy (BLE), and Ultra-Wideband (UWB) may all be applied.

According to an embodiment of the present specification, the probability of transmission of infectious disease and the like can be determined much faster than the conventional method using video information such as CCTV. Therefore, it has the effect of reducing the speed and scope of the spread of a catastrophe (for example, an epidemic, and the like).

The method according to an example of the present specification may be performed on the premise of the following.

1. People with potential exposure to disaster, such as a disease or the like, are using one or more smart devices.

2. The relevant authorities (Ministry of Science and Technology Information and Communication, and the like) have information about these smart devices (for example, phone number, International Mobile Subscription Identifier (IMSI), Wi-Fi medium access control (MAC) Address, and the like).

3. These smart devices may have application functions that provide Location-based Services and Proximity Services.

4. The application can operate in conjunction with the wireless access technology.

5. The application can be operated automatically or with the consent of the smart device user when a certain situation occurs.

Hereinafter, a method for preventing the spread of infectious disease will be described.

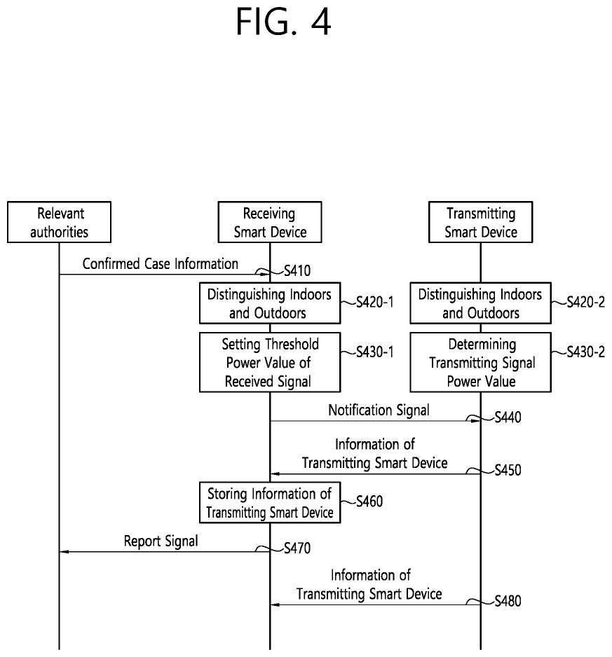

FIG. 4 is a diagram illustrating a method for preventing the spread of infectious disease according to an embodiment of the present specification.

Referring to FIG. 4, the relevant authorities can identify a person who is potentially capable of transmitting the infection and activate the application by using the smart device information of the person who can potentially spread the infection. For example, the relevant authorities may operate the application of the smart device of the person capable of spreading the infection. For example, the relevant authorities may operate the application on all smart devices in which the application is installed as well as on the person capable of spreading the infection.

Personal privacy issues may be accompanied, but this is not discussed in this specification. Legislative bodies (for example, the National Assembly) can make provisions for this situation. For example, when the World Health Organization (WHO) declares a global pandemic, it can be activated.

For example, by providing smart device users with an opportunity to select “Opt-in” or “Opt-out”, it can also induce the user's voluntary application activation. For example, the application may operate only when the user approves it.

If the outbreak area is outside the country, the Ministry of Justice, if the outbreak area is inside the country, the Ministry of Public Administration and Security, and the like may screen visitors to the affected area (for example, visitors are selected based on entry/exit records, local visit records, etc.) and notify other relevant organizations (for example, Ministry of Science and Technology Information and Communication, Ministry of Health and Welfare, etc.). For example, the Ministry of Science and Technology Information and Communication can derive smart device information of a person who is likely to be infected through the notified personal information.

The relevant authorities may provide information on the confirmed case to the receiving smart device (S410). For example, the Ministry of Health and Welfare may provide information according to medical conditions. For example, the Ministry of Health and Welfare can provide information on the contact radius according to the characteristics of the epidemic, such as a radius of 3 meters for the COVID-19 virus and 1 meter for the Ebola virus for the indoor. Another information may be information related to contact time. For example, the Ministry of Health and Welfare can provide information that there is a possibility of infection when there is close contact for at least 1 minute within a radius of 3 meters, or contact for more than 3 minutes within a radius of 1 meter outdoors. The contact radius (for example, 1 meter, 3 meters) and the contact time (for example, 1 minute, 3 minutes) described above are examples, and are not limited thereto, and may be set variously.

Indoor and outdoor information may be set differently. Location-based service can be used to distinguish between indoor and outdoor. For example, smart devices to which the application is activated can identify and register the current location by using location-based services provided by Wi-Fi, BLE, UWB, and the like in an indoor case, and by using Cellular-based Location Services in an outdoor case. For example, the smart device may determine whether it is currently located indoors or outdoors by using a global positioning system (GPS), a WLAN signal, or the like. For example, based on the reception strength of the GPS signal, the smart device may determine that it is indoors when the reception strength falls below a predetermined threshold value.

Based on the provision of information from medical-related institutions, the institution in charge of communication can set the operating parameters of the application according to the situation. The set parameters can be propagated to each mobile communication operator, cable operator, Internet operator, and the like. That is, the receiving smart device may obtain information about the operation parameter in various ways.

Accordingly, the transmitting/receiving smart device may perform an operation for classifying indoor/outdoor (S420-1/2).

For example, in the case of the COVID-19 virus, the receiving smart device may acquire parameter information corresponding to the COVID-19 virus, and may acquire smart device information within a contact radius (for example, 3 meters) according to a condition (that is, a condition according to the parameter).

The operation parameters may include transmission power, a transmission period, a threshold for the number of responses of the same device within a predetermined time, and the like.

Smart devices with activated applications can identify the current location and register by using Location-based Services provided by Wi-Fi, BLE, UWB, and the like in an indoor case, and by using Cellular-based Location Services in an outdoor case. An application can acquire information about a nearby smart device using the Proximity Service (BLE, Wi-Fi Direct, Wi-Fi Aware, and the like) within the range defined in the parameter. The acquisition information may include a phone number, an International Mobile Subscription Identifier (IMSI), a Wi-Fi MAC Address, and the like.

For example, proximity-based services may be an operation in which a smart device broadcasts a beacon with transmission power capable of reaching other smart devices within 3 meters of the smart device.

Alternatively, in the case of Wi-Fi (that is, WLAN), the smart device can transmit the FTM (Fine Timing Measurement) signal by setting the transmission power so that other smart devices within 3 meters can receive it.

Transmission power may vary depending on the environment. For example, the transmission power to reach 3 meters in an indoor open hall may be higher than the transmission power to reach 3 meters in a cubicle environment. That is, even indoors, the transmission power may be set differently depending on the environment. For example, the smart device may obtain information about the shape of the current location, and may determine transmission power based on the information.

The transmitting smart device may determine the transmit signal power value (S430-2).

Also, the transmission power may vary according to the outdoor/indoor environment. For example, since the level of the ambient interference signal may be increased outdoors, the transmission power may be set higher. For example, the transmission power value may be set differently depending on the indoor structure, even in the same indoor case.

The receiving smart device may configure a threshold power value of the received signal (S430-1).

In addition, the receiving smart device may also configure a threshold power value for the received signal. For example, the receiving smart device may set a threshold power value according to the surrounding environment and decode only the received signal exceeding the threshold power value. For example, the threshold power value may be set differently depending on the indoor/outdoor environment, and may be set differently depending on the indoor structure, even in the same indoor case.

Indoor location information (for example, indoor map) can be used as data that can cause such a transmission power change. Such information may be provided to the smart device by the side that manages the indoor environment.

The receiving smart device may transmit a notification signal (S440). For example, the receiving smart device may transmit a notification signal to nearby transmitting smart devices. In response to a Broadcast Beacon, Unicast, or Multicast transmission (i.e., a notification signal), smart devices within the contact radius can activate the application and transmit smart device related information to the smart device that performed the broadcast.

The transmitting smart device may transmit information of the transmitting smart device (S450). For example, since there may be privacy issues, the responding device (that is, the transmitting smart device) may request the user to determine whether to allow transmission. For example, when a receiving smart device sends a notification signal to a nearby transmitting smart device through a beacon, the transmitting smart device may make a decision on whether to transmit its own information. That is, the user of the transmitting smart device may decide whether to consent to provide its own information. Therefore, the transmitting smart device may or may not transmit its own information based on the user's response.

Such operations may continue depending on the location change of the smart device. For example, when a smart device moves from indoors to outdoors, the smart device can automatically update the setting parameter values (for example, transmission power, number of transmissions, critical power for received signals, and the like) through the transformation of parameters.

The receiving smart device may store information of the transmitting smart device (S460). For example, the smart device may acquire and store information corresponding to information provided by a competent authority. For example, the stored information may be automatically deleted after a certain period of time. For example, in the case of the COVID-19 virus, information that has passed a certain period (for example, 14 days) may be automatically deleted.

The storage condition can be set based on the number of responses within a certain time. For example, if the transmission period is once per second, information about a smart device that has responded more than 50 times in 3 minutes may be stored. That is, the smart device may store information of the smart device included in the signal received a predetermined number of times or more within a predetermined time.

For example, the smart device may transmit the stored information of other smart devices at the request of a related authority, periodically, or in a specific situation.

For example, the relevant authorities can broadcast the information of the infected confirmed case through the telecommunication company (wired or wireless). For example, the receiving smart device may acquire the information of the infected confirmed case through the telecommunication company.

The receiving smart device may transmit a report signal (S470). For example, the receiving smart device may upload related information (contact time, location, and the like) when information (phone number, MAC ID, and the like) matching the broadcast of the authority is stored. For example, when information matching the information on the confirmed case exists among the stored smart device information, the receiving smart device may transmit a report signal including information of the matching smart device to the server (or terminal) of the relevant authority.

Based on the information received (that is, the report signal), the relevant authorities can derive a list of contacts with a confirmed infected case, and can track and centrally manage the infected person and their contacts.

The receiving smart device may receive information on the transmitting smart device again (S480). For example, the receiving smart device may ignore the information of the transmitting smart device obtained through this operation, if it already exists in relation to this emergency. That is, the smart device may store the information of the other smart device that has already been received, and when a signal including information corresponding to the previously stored information is received again, it may be ignored. For example, when a smart device receives information from another smart device, it can monitor how many times the information is received within a specific threshold time. When the information of the other smart device is information of a smart device that has already been stored, the monitoring operation may not be performed.

For example, since power and resources may be wasted when smart device information of a family living together is repeatedly re-received, the smart device may ignore a signal including already stored information.

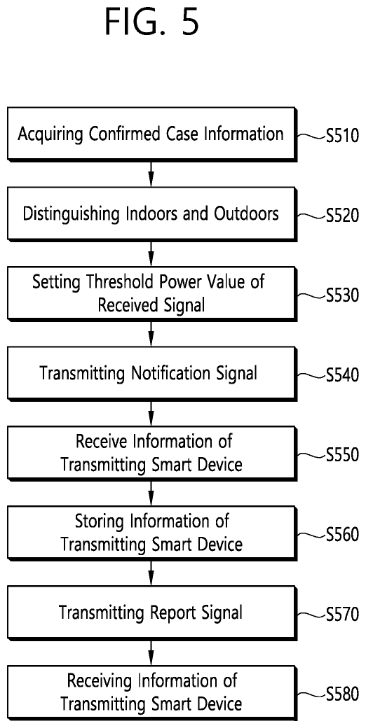

FIG. 5 is a diagram illustrating an embodiment of a method for operating a receiving smart device.

Referring to FIG. 5, the receiving smart device may acquire information about the confirmed case (S510). For example, the receiving smart device may obtain the confirmed case information from the relevant authorities. For example, the information about confirmed cases may include at least one of a medium access control (MAC) address of a smart device used by the confirmed cases, phone number information of the confirmed cases, and information related to criteria for judging a person in contact with the confirmed cases.

The receiving smart device may distinguish indoors and outdoors (S520). For example, the receiving smart device may distinguish whether the place where the receiving smart device is located is indoors or outdoors.

The receiving smart device may set a threshold power value of the received signal (S530). For example, the receiving smart device may set a threshold power value of the first signal for receiving the first signal to a first value if the place where the receiving smart device is located is indoors, and may set a threshold power value of the first signal for receiving the first signal to a second value if the place where the receiving smart device is located is outdoors. For example, the first value may be smaller than the second value.

The receiving smart device may transmit a notification signal (S540). For example, the notification signal may be a broadcast beacon or a unicast/multicast signal. The notification signal may be a signal for the receiving smart device to request information of the transmitting smart device from the transmitting smart device.

The receiving smart device may receive information of the transmitting smart device (S550). For example, the receiving smart device may receive, from the transmitting smart device, a first signal including the first information of the transmitting smart device. For example, the strength of the first signal may vary depending on whether the transmitting smart device is located indoors or outdoors.

The receiving smart device may store information of the transmitting smart device and may transmit a report signal (S560, S570). For example, based on the first information included in the confirmed case information, the receiving smart device may transmit a report signal including the first information and location information at which the first signal was received. For example, the report signal may be transmitted when the receiving smart device receives the first signal more than a threshold number of times within a first threshold period, and the receiving smart device may store the first information included in the report signal.

The receiving smart device may receive information of the transmitting smart device (S580). For example, the receiving smart device may receive the second signal including the first information from the transmitting smart device. For example, the receiving smart device may ignore the second signal, based on that the first information included in the second signal has already been stored.

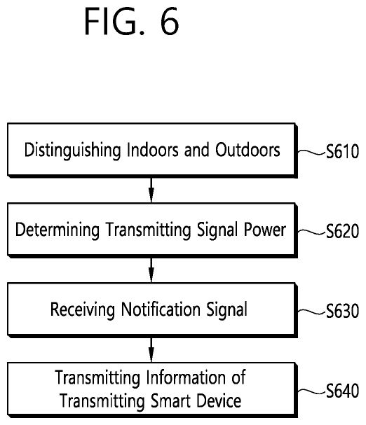

FIG. 6 is a diagram illustrating an embodiment of a method of operating a transmitting smart device.

Referring to FIG. 6, the transmitting smart device may distinguish indoors and outdoors (S610). For example, the transmitting smart device may distinguish whether the place where the transmitting smart device is located is indoors or outdoors.

The transmitting smart device may determine the transmitting signal power (S620). For example, the transmitting smart device may set the transmission power of the first signal to a first value if the place where the transmitting smart device is located is indoors, and may set the transmission power of the first signal to a second value when the place where the transmitting smart device is located is outdoors. For example, the first value may be smaller than the second value.

The transmitting smart device may receive a notification signal (S630). For example,

The transmitting smart device may transmit information of the transmitting smart device (S640). For example, the notification signal may be a broadcast beacon or a unicast/multicast signal. The notification signal may be a signal for the receiving smart device to request information of the transmitting smart device from the transmitting smart device.

Some of the detailed steps shown in the example of FIGS. 5 and 6 may be omitted. In addition to the steps shown in FIGS. 5 and 6, other steps may be added, and the order of the steps may vary. Some of the above steps may have their own technical meaning.

The technical features of the present specification described above may be applied to various devices and methods. For example, the above-described technical features of the present specification may be performed/supported through the apparatus of FIGS. 1 and/or 3. For example, the above-described technical features of the present specification may be applied only to a part of FIGS. 1 and/or 3. For example, the technical features of the present specification described above are implemented based on the processing chips 114 and 124 of FIG. 1, or implemented based on the processors 111 and 121 and the memories 112 and 122 of FIG. 1, or, may be implemented based on the processor 310 and the memory 320 of FIG. 3. For example, a device of the present disclosure comprises, a memory; and a processor operably coupled to the memory, wherein the processor is configured to: acquire information about confirmed cases; receive, from a transmitting smart device, a first signal including first information of the transmitting smart device; and transmit a report signal including the first information and location information at which the first signal was received, based on the first information being included in the information about confirmed cases.

The technical features of the present specification may be implemented based on a computer readable medium (CRM). For example, at least one computer readable medium, proposed by the present specification, storing instructions which, based on being executed by at least one processor of an access point (AP) multi-link device (MLD) in a wireless local area network system, perform operations, the operations comprise: acquiring, by the receiving smart device, information about confirmed cases; receiving, by the receiving smart device from a transmitting smart device, a first signal including first information of the transmitting smart device; and transmitting, by the receiving smart device, a report signal including the first information and location information at which the first signal was received, based on the first information being included in the information about confirmed cases.

The instructions stored in the CRM of the present specification may be executed by at least one processor. At least one processor related to CRM in the present specification may be the processors 111 and 121 or the processing chips 114 and 124 of FIG. 1, or the processor 310 of FIG. 3. Meanwhile, the CRM of the present specification may be the memories 112 and 122 of FIG. 1, the memory 320 of FIG. 3, or a separate external memory/storage medium/disk.

The foregoing technical features of this specification are applicable to various applications or business models. For example, the foregoing technical features may be applied for wireless communication of a device supporting artificial intelligence (AI).

Artificial intelligence refers to a field of study on artificial intelligence or methodologies for creating artificial intelligence, and machine learning refers to a field of study on methodologies for defining and solving various issues in the area of artificial intelligence. Machine learning is also defined as an algorithm for improving the performance of an operation through steady experiences of the operation.

An artificial neural network (ANN) is a model used in machine learning and may refer to an overall problem-solving model that includes artificial neurons (nodes) forming a network by combining synapses. The artificial neural network may be defined by a pattern of connection between neurons of different layers, a learning process of updating a model parameter, and an activation function generating an output value.

The artificial neural network may include an input layer, an output layer, and optionally one or more hidden layers. Each layer includes one or more neurons, and the artificial neural network may include synapses that connect neurons. In the artificial neural network, each neuron may output a function value of an activation function of input signals input through a synapse, weights, and deviations.

A model parameter refers to a parameter determined through learning and includes a weight of synapse connection and a deviation of a neuron. A hyper-parameter refers to a parameter to be set before learning in a machine learning algorithm and includes a learning rate, the number of iterations, a mini-batch size, and an initialization function.

Learning an artificial neural network may be intended to determine a model parameter for minimizing a loss function. The loss function may be used as an index for determining an optimal model parameter in a process of learning the artificial neural network.

Machine learning may be classified into supervised learning, unsupervised learning, and reinforcement learning.

Supervised learning refers to a method of training an artificial neural network with a label given for training data, wherein the label may indicate a correct answer (or result value) that the artificial neural network needs to infer when the training data is input to the artificial neural network. Unsupervised learning may refer to a method of training an artificial neural network without a label given for training data. Reinforcement learning may refer to a training method for training an agent defined in an environment to choose an action or a sequence of actions to maximize a cumulative reward in each state.

Machine learning implemented with a deep neural network (DNN) including a plurality of hidden layers among artificial neural networks is referred to as deep learning, and deep learning is part of machine learning. Hereinafter, machine learning is construed as including deep learning.

The foregoing technical features may be applied to wireless communication of a robot.

Robots may refer to machinery that automatically process or operate a given task with own ability thereof. In particular, a robot having a function of recognizing an environment and autonomously making a judgment to perform an operation may be referred to as an intelligent robot.

Robots may be classified into industrial, medical, household, military robots and the like according uses or fields. A robot may include an actuator or a driver including a motor to perform various physical operations, such as moving a robot joint. In addition, a movable robot may include a wheel, a brake, a propeller, and the like in a driver to run on the ground or fly in the air through the driver.

The foregoing technical features may be applied to a device supporting extended reality.

Extended reality collectively refers to virtual reality (VR), augmented reality (AR), and mixed reality (MR). VR technology is a computer graphic technology of providing a real-world object and background only in a CG image, AR technology is a computer graphic technology of providing a virtual CG image on a real object image, and MR technology is a computer graphic technology of providing virtual objects mixed and combined with the real world.

MR technology is similar to AR technology in that a real object and a virtual object are displayed together. However, a virtual object is used as a supplement to a real object in AR technology, whereas a virtual object and a real object are used as equal statuses in MR technology.

XR technology may be applied to a head-mount display (HMD), a head-up display (HUD), a mobile phone, a tablet PC, a laptop computer, a desktop computer, a TV, digital signage, and the like. A device to which XR technology is applied may be referred to as an XR device.

The claims recited in the present specification may be combined in a variety of ways. For example, the technical features of the method claims of the present specification may be combined to be implemented as a device, and the technical features of the device claims of the present specification may be combined to be implemented by a method. In addition, the technical characteristics of the method claim of the present specification and the technical characteristics of the device claim may be combined to be implemented as a device, and the technical characteristics of the method claim of the present specification and the technical characteristics of the device claim may be combined to be implemented by a method.

1. A method performed by a receiving smart device, the method comprising:
acquiring, by the receiving smart device, information about confirmed cases;
receiving, by the receiving smart device from a transmitting smart device, a first signal including first information of the transmitting smart device; and
transmitting, by the receiving smart device, a report signal including the first information and location information at which the first signal was received, based on the first information being included in the information about confirmed cases.

2. The method of claim 1,
wherein the information about confirmed cases includes at least one of a medium access control (MAC) address of a smart device used by the confirmed cases, phone number information of the confirmed cases, and information related to criteria for judging a person in contact with the confirmed cases.

3. The method of claim 1,
wherein a strength of the first signal varies depending on whether the transmitting smart device is located indoors or outdoors.

4. The method of claim 1, wherein the method further comprises,
determining, by the receiving smart device, whether a place where the receiving smart device is located is indoors or outdoors;
configuring, by the receiving smart device, a threshold power value of the first signal for receiving the first signal to a first value, when the place where the receiving smart device is located is indoors; and
configuring, by the receiving smart device, a threshold power value of the first signal for receiving the first signal to a second value, when the place where the receiving smart device is located is outdoors,
wherein the first value is less than the second value.

5. The method of claim 1,
wherein the report signal is transmitted, when the receiving smart device receives the first signal more than a threshold number within a first threshold period, and
wherein the method further comprising,
storing, by the receiving smart device, the first information included in the report signal.

6. The method of claim 5, wherein the method further comprising,
receiving, by the receiving smart device from the transmitting smart device, a second signal including the first information; and
ignoring, by the receiving smart device, the second signal, based on that the first information included in the second signal has already been stored.

7. The method of claim 5, wherein the method further comprising,
deleting, by the receiving smart device, the first information based on the second threshold time elapsed since the first information was stored.

8. A receiving smart device, comprising:
a transceiver for transmitting and receiving a radio signal; and
a processor coupled to the transceiver, wherein the processor is configured to:
acquire information about confirmed cases;
receive, from a transmitting smart device, a first signal including first information of the transmitting smart device; and
transmit a report signal including the first information and location information at which the first signal was received, based on the first information being included in the information about confirmed cases.

9. The receiving smart device of claim 8,
wherein the information about confirmed cases includes at least one of a medium access control (MAC) address of a smart device used by the confirmed cases, phone number information of the confirmed cases, and information related to criteria for judging a person in contact with the confirmed cases.

10. The receiving smart device of claim 8,
wherein a strength of the first signal varies depending on whether the transmitting smart device is located indoors or outdoors.

11. The receiving smart device of claim 8, wherein the processor is further configured to:
determine whether a place where the receiving smart device is located is indoors or outdoors;
configure a threshold power value of the first signal for receiving the first signal to a first value, when the place where the receiving smart device is located is indoors; and
configure a threshold power value of the first signal for receiving the first signal to a second value, when the place where the receiving smart device is located is outdoors,
wherein the first value is less than the second value.

12. The receiving smart device of claim 8,
wherein the report signal is transmitted, when the receiving smart device receives the first signal more than a threshold number within a first threshold period, and
wherein the receiving smart device stores the first information included in the report signal.

13. The receiving smart device of claim 12, wherein the processor is further configured to:
receive, from the transmitting smart device, a second signal including the first information; and
ignore the second signal, based on that the first information included in the second signal has already been stored.

14. The receiving smart device of claim 12, wherein the processor is further configured to:
delete the first information based on the second threshold time elapsed since the first information was stored.

15. A method performed by a transmitting smart device, the method comprising:
determining, by the transmitting smart device, whether a place where the transmitting smart device is located is indoors or outdoors;
configuring, by the transmitting smart device, a transmit power of a first signal to a first value, when the place where the transmitting smart device is located is indoors; and
configuring, by the transmitting smart device, a transmit power of the first signal to a second value, when the place where the transmitting smart device is located is outdoors,
wherein the first value is less than the second value.

16.-18. (canceled)
